(19) United States
(12) Patent Application Publication
(10) Pub. No.: US 2002/0065529 A1
(43) Pub. Date: May 30, 2002

(54) Vascular occlusion device with adjustable length

(75) Inventors: Alexandre Laurent, Courbevoie (FR); Laurent Domas, Paris (FR)

Correspondence Address: FITCH EVEN TABIN AND FLANNERY, 120 SOUTH LA SALLE STREET, SUITE 1600, CHICAGO, IL, 60603-3406, US

(73) Assignee: Assistance Publique-Hopitaux De Paris

(21) Appl. No.: 09/846,894

(22) Filed: May 1, 2001

Related U.S. Application Data
(63) Continuation of application No. PCT/FR99/02671, filed on Nov. 2, 1999.

(30) Foreign Application Priority Data
Nov. 2, 1998 (FR) 9813749

(51) Int. Cl. A61B 17/08

(52) U.S. Cl. 606/151

(57) ABSTRACT

The invention relates concerns electrolytically detachable biocompatible metal wire turns and a conductor guide compatible in shape and size with their axial translation displacement in a catheter. Said turns and devices are designed for setting electrically detachable vascular occlusion turns with adjustable length.

### FIELD OF THE INVENTION

The present invention relates to a biocompatible metal coil and to devices for positioning adjustable length electroseverable vascular occlusion devices.

### BACKGROUND OF THE INVENTION

Vascular occlusion coils are used to occlude a variety of pathological processes such as aneurysms (vascular ectasia), arteriovenous fistulas or to occlude arteries connected with pathological processes (in particular tumours and haemorrhages).

In order to produce a vascular occlusion by modifying the flow conditions of the blood in an aneurysm, fistula or vessel, coils are constituted by a metal wire prepared in the form of a coil with secondary shape memory which means that, once in position, they can form three-dimensional geometric shapes.

These vascular occlusion coils are usually positioned using a catheter. Such coils have a pre-determined length and are positioned using a guidewire-pusher, the guidewire-pusher pushing the coil inside the catheter, the guidewire and coil being independent of each other.

The use of a coil connected to the guidewire by a connection comprising a mechanical detachment system is also known. Such mechanical detachment devices are known from International patent applications WO-A-93/11719 and WO-A-93/11825. Detachment by laser fusion of the connection piece or by electrolysis of the connection piece is also known.

In general, in all of the known devices, the length of the coil is pre-determined, which has the following major disadvantage: when the length of the coil is unsuitable, in particular if it is too long, it has to be withdrawn.

### SUMMARY OF THE INVENTION

The invention concerns a wire coil formed from an electroseverable biocompatible metal material. The coils can be used for vascular occlusion.

The coils of the invention overcome the major disadvantage mentioned above. After positioning the coil in the vascular cavity, the coil is severed to the desired length. It is not simply detached by being pushed from the catheter, by mechanical detachment or by melting a joining piece. In particular, it is distinct from the electric detachment that is known and described in particular in U.S. Pat. No. 5,122,136, i.e., electrocorrosion is not used to dissolve a joining piece but to cut the coil itself to the desired length. The coils described in U.S. Pat. No. 5,122,136 are constituted by a material that is not susceptible to being either disintegrated or corroded in the blood by the electrocorrosion methods employed. With the coils of the invention, the vascular cavity can be filled with a single coil, or the same coil can be used several times for successive cuts. The coil length is not pre-determined but can be adjusted to the pathological process.

### DESCRIPTION OF THE PREFERRED EMBODIMENTS OF THE INVENTION

The term “electroseverable metal material” as used in the invention means any conductive material that can be severed by an electrochemical method that is compatible with electrolytic methods applied to living organisms, in particular to the human body.

Examples of electroseverable metal materials that can be used in human implantation that can be cited are 316L steel, 316LVM steel and nickel-titanium (Ni-Ti) 55/45 alloys.

In accordance with the invention, the diameter of the wire is in the range 0.01 millimetres (mm) to 0.5 mm, preferably in the range 0.05 mm to 0.1 mm, and the diameter of the coil is in the range 0.05 mm to 5 mm, preferably in the range 0.2 mm to 1 mm.

Preferably, the coil thus has dimensions that are suitable for it to be translated through a catheter of dimensions suitable for reaching the occlusion site.

The coil is radioopaque.

In accordance with the invention, the biocompatible metal material is selected from materials that can be endowed with shape memory or which have shape memory. The metals cited above can be used to produce wires of a diameter suitable for the production of primary coils and secondary shapes, the secondary geometric shape(s) forming in the cavity after detachment.

To produce such coils, a wire with a diameter suitable for the desired coil is wound around a mandrel with a diameter suitable for the desired coil. Depending on the material, a heat treatment is optionally carried out to set the primary coil shape. Ni-Ti or steel wires are set in the primary shape by a heat treatment carried out during or after winding. To obtain the secondary shapes, for example the shape of a secondary coil, at another time the coils can themselves be wound around a further mandrel and can optionally be annealed.

Further, the invention concerns a device comprising a coil in accordance with the invention and an electrically conductive guidewire with a shape and length that is compatible with its axial translational displacement in a catheter. The distal end of the guidewire is integral with the proximal end of the coil via a conductive securing means.

Regarding the guidewire, the conductive guidewires described in U.S. Pat. No. 5,122,136 can be cited.

The distal end of the guidewire is tapered, for example conical. It can be introduced into the proximal end of the coil. Further, the guidewire and coil are connected via a conductive securing means forming a connection.

In a particular embodiment, the coil is fixed to the guidewire by means of a conductive weld or soldered joint. In this case, the soldered joint is preferably coated with a coating that insulates the securing means or connection between the coil and the guidewire from the external medium. As an example, this coating can be constituted by medical grade polymers (silicone, PTFE, a heat shrinkable polymer sleeve) or by “hydrophilic” gels that are known and usually used to coat catheters and medical guidewires, in particular vascular guidewires.

In a second embodiment, the invention concerns a device comprising a coil in accordance with the invention wherein a portion that is remote from its proximal end is coated with an insulating sleeve or gel and forms a guidewire acting as a mechanical guidewire and as a conductor for current insulated from the medium.

Thus the invention provides a device comprising a coil and a conductive guidewire of a shape and length that is compatible with its axial translational displacement in a catheter, characterized in that it comprises a coil in accordance with the invention and a coating insulating it from the external medium over at least a portion of the coil, that portion forming a guidewire.

In this embodiment, the portion of the coil forming the guidewire is de facto integral with the portion of the coil intended to be detached, in one or more segments. This is the same coil that is electroseverable over a first portion from its end distal to its proximal end, and is coated with an insulator from this proximal end, which is also the distal end of the portion thereof forming the guidewire.

The first function of the guidewire is to guide the coil in the catheter and beyond in the vascular lumen. The fact that the guidewire and coil are integral means that forward and reverse movements can be made, along with twisting movements without the risk of blockage, nor the risk of breaking or folding of the coil to be detached. Further, the guidewire, which is conductive, is compatible with the coil electrocorrosion process, i.e., it can resist electrocorrosion because of its nature or its coating. It can, for example, be constituted by a non-corrosive conductive material or it can be coated with a sleeve and/or an insulating gel as mentioned above.

Further, the invention provides a device for positioning vascular occlusion coils compatible with its use in combination with a catheter and an electric current generator, comprising a coil of a wire of an electroseverable biocompatible metal material compatible with its positioning in a vascular cavity and a conductive guidewire compatible with displacement of the coil and the guidewire in axial translation in the catheter, the distal end of said guidewire being secured to the proximal end of the coil, characterized in that the coil is electroseverable from its proximal end to its distal end, in that the guidewire can be connected via its proximal end to the generator and in that the guidewire and coil are secured together in a conductive manner.

The invention also provides a device for positioning a vascular occlusion coil compatible with its use in combination with a catheter and an electric current generator, comprising a coil compatible with its positioning in a vascular cavity and a conductor guidewire compatible with displacement of the coil and guidewire in axial translation in the catheter, the distal end of said guidewire being secured to the proximal end of the coil, characterized in that the coil is formed from wire of an electroseverable biocompatible metal material from its proximal end to its distal end, in that the guidewire can be connected via its proximal end to the generator and in that the guidewire is a portion of the coil coated with an insulating coating.

The catheters and electric current generators are known and have been described, in particular in U.S. Pat. No. 5,122,136.

Finally, the following method is used. A catheter is introduced into the blood vessel so that its distal end reaches the cavity to be occluded. The coil-guidewire assembly is introduced into the catheter. The desired length of coil is pushed into the cavity to be occluded. The guidewire is then connected to the generator that is connected to a counter-electrode applied to the patient's skin. The electric current is applied and electrocorrosion occurs. The segment of coil of the desired length is then detached. One (or more) other segment(s) of the same coil can be detached by one (or more) successive operation(s) without the need to position a new catheter or a further coil-guidewire device.

### EXAMPLE 1

316L stainless steel wires satisfying AISI standard 316, W N⁰1.4401 and steel wires satisfying ISO standard 5832/1 grade D, DIN17443 and ASTM-F138 grade 2, also austenitic nickel/titanium (Ni-Ti) alloy wires in proportions of 55/45 with a diameter of 70, 100, 125, 150 μm were wound by mechanical winding around a mandrel into coils with a diameter of 0.2 to 1 mm to produce a coil.

After winding, a heat treatment was carried out to produce the primary set.

### EXAMPLE 2

In Vitro Electrocorrosion

The metal wire (316L steel, 316LVM steel, Ni-Ti 55/45 with diameter d: 0.07; 0.1; 0.125; 0.150 mm) was introduced into a coaxial microcatheter 20 cm long and protruding from it by a length 1: 1, 1.5, 2, 3, 5, 10, 20 cm and was immersed in a solution of artificial plasma (Hanks solution). A counter-electrode was also immersed in the medium. It was a Hg/Hg₂-SO_{4/}K₂SO₃ electrode.

The current was applied using a potentiostat-galvanostat (Tacussel PGS 201 T analytical radiometer) to produce intensities of 0.1 mA to 10 A. Intensities of 1, 3, 4, 5, 7, 8 and 9 milliamperes were applied.

It was observed that break occurred in the zone of the wire emerging from the catheter. There was no corrosion phenomenon upstream or downstream of the break point.

The time to break was measured and is shown in Tables I and II below. The time to break was shorter as the diameter was reduced.

It was observed that for a given diameter d, the time to break was independent of the length 1 and only slightly dependent on the current intensity applied.

TABLE I

| | Time to break |
|---|---|
| Material⁽*⁾ | (mean and standard deviation) |
| Austenitic Ni—Ti | 217 ± 203 seconds |
| 55/45 | |
| 316L steel | 286 ± 189 seconds |
| 31 6LVM steel | 380 ± 180 seconds |

⁽*⁾d = 0.1 mm

TABLE II

| | Time to sever |
|---|---|
| Diameter⁽**⁾ | (sec) |
| d (mm) | (mean and SD) |
| 0.07 | 171 seconds ± 65 |
| 0.1 | 290 seconds ± 196 |
| 0.125 | 627 seconds ± 112 |
| 0.150 | 569 seconds ± 79 |

⁽**⁾316L steel

### EXAMPLE 2

Bis

Measurements analogous to those of Example 2 were carried out using Ni-Ti 55/45 wires with a diameter d=0.07; 0.10; and 0.15 mm.

The following in vitro results were obtained for time t:

| Ni—Ti 55/45 wire | Time to sever | | Min | Max |
|---|---|---|---|---|
| d(mm) | (s) | No of tests | (s) | (s) |
| 0.07 | 173.5 ± 177.5 | 64 | 31 | 600 |
| 0.10 | 222.2 ± 189.2 | 104 | 49 | 760 |
| 0.15 | 627.2 ± 127.9 | 22 | 600 | 1200 |

### EXAMPLE 3

In Vivo Electrocorrosion

A 100 cm microcatheter was introduced percutaneously into the femoral artery of an anaesthetised rabbit and its end was positioned super-renally in the aorta. The counter-electrode was a silver skin electrode. The wire was introduced into the microcatheter, its distal end protruding from the distal end of the catheter by about 10 cm. This end was exposed to the blood flow.

The current was applied under the same conditions as in Example 2 (in vitro).

Breakage was observed to occur in vivo in the same manner as in vitro and the time to break was slightly dependent on the medium (in vitro or in vivo) and shown in Table III below.

TABLE III

| | Time to section | Time to section |
|---|---|---|
| | In vitro | In vivo |
| Material⁽***⁾ | (s) | (s) |
| Austenitic nickel-titanium | 170 ± 117 | 295 ± 278 |
| 55/45 | | |
| 316L | 447 ± 201 | 350 ± 173 |
| 316LVM | 377 ± 214 | 383 ± 135 |

***⁾d = 0.1 mm

### EXAMPLE 4

In Vitro Corrosion

316L and Ni-Ti 55/45 metal coils with a diameter of 0.3 mm and a wire diameter of 0.05 mm were immersed in an artificial plasma solution (Hanks solution). A counter-electrode was also immersed in the medium. The current was applied using a potentiostat-galvanostat (Tacussel PGS 201 analytical radiometer). Intensities of 2 milliamperes were applied. break took place in the zone where the wire emerged from the catheter. The time to break was measured and is shown in Table IV below. It can be seen that the time to break was independent of the length immersed in the solution.

TABLE IV

| Coil | Time to sever | | Min | Max |
|---|---|---|---|---|
| material | (s) | No of tests | (s) | (s) |
| 316L | 428 ± 112.9 | 24 | 290 | 600 |
| Ni—Ti | 458 ± 46.5 | 70 | 331 | 5242 |

### EXAMPLE 5

In Vivo and In Vitro Electrocorrosion of Ni-Ti 55/45 Coils

Ni-Ti coils with a diameter of 0.05 mm for the wire and 0.3 mm for the coil were used.

In vitro electrocorrosion was studied using the technique described in Example 4; in vivo electrocorrosion was studied using the technique described in Example 3.

For introduction into the catheter, the coil was prepared as follows: a stainless steel rod was introduced into the proximal end of the coil, the two pieces were assembled coaxially using a drop of acrylic adhesive. The coil was introduced into the microcatheter and pushed by its guidewire. The length of the coil exposed to the blood flow was determined by radiological monitoring, or by measuring the length of the guidewire introduced into the microcatheter. The time to break was measured (see Table V). it was observed that the time to break in vivo and in vitro was independent of the length of the coil exposed to the blood flow or to the medium.

TABLE V

| | Time to break | | | |
|---|---|---|---|---|
| | (s) | | | |
| | Mean & SD | No of tests | Min | Max |
| Vitro | 472.9 ± 37.9 | 39 | 331 | 542 |
| Vivo | 439.3 ± 50.0 | 31 | 345 | 519 |

What is claimed is:

1. A wire coil formed from an electroseverable biocompatible metal wire material, wherein said electroseverable biocompatible metal wire material is a 55/45 to 45/55 nickel-titanium alloy.

2. The wire coil according to claim 1, wherein a diameter of said wire is in the range of 0.001 mm to 0.5 mm, the diameter of the wire coil is in the range of 0.02 mm to 5 mm.

3. The wire coil according to claim 1, wherein a diameter of said wire is in the range of 0.005 mm to 0.1 mm and the diameter of the wire coil is in the range of 0.2 mm to 1 mm.

4. A device comprising said wire coil formed from an electroseverable biocompatible metal wire material selected from 316L steel, 316LVM steel and a 55/45 to 45/55 nickel-titanium alloy and a conductive guidewire with a shape and length that is compatible with its axial translational displacement in a catheter, the distal end of the guidewire being secured to the proximal end of the wire coil via a conductive securing means.

5. The device according to claim 4, wherein the conductive securing means is a conductive weld or a soldered joint.

6. The device according to claim 4, wherein the securing means has a coating that insulates said securing means from an external medium.

7. A device comprising a wire coil and a conductive guidewire of a shape and length that is compatible with its axial translational displacement in a catheter, comprising a wire coil according to claim 1 and a coating that insulates said wire coil from an external medium over at least a portion of said wire coil, said portion forming a guidewire.

8. The device according to claim 7, wherein the coating that insulates said wire coil comprises a sleeve and/or a gel.

9. A device for positioning a vascular occlusion coil in combination with a catheter and an electric current generator, comprising a wire coil of an electroseverable biocompatible metal material and a conductive guidewire compatible with displacement of the wire coil and the guidewire in axial translation in the catheter, the distal end of said guidewire being secured to the proximal end of the coil, wherein the wire coil is electroseverable from its proximal end to its distal end, wherein the conductive guidewire is connected via its proximal end to the generator and wherein the guidewire and said wire coil are secured together in a conductive manner.

10. A device for positioning a vascular occlusion coil in combination with a catheter and an electric current generator, comprising a coil and a conductive guidewire compatible with displacement of the coil and guidewire in axial translation in the catheter, the distal end of said guidewire being secured to the proximal end of the coil, wherein the coil is formed from a wire of an electroseverable biocompatible metal material from its proximal end to its distal end, wherein the guidewire can be connected via its proximal end to the generator and wherein the guidewire is a portion of the wire coil coated with an insulating coating.

11. A method for positioning a vascular occlusion wire coil, said method comprising:
(a) introducing a catheter into a blood vessel;
(b) introducing into the catheter an assembly comprising said wire coil formed from an electroseverable biocompatible metal material and a guidewire;
(c) pushing said wire coil to the desired length; and
(d) connecting the guidewire to a generator; and applying a vascular electric current.

12. The method according to claim 11, wherein said wire coil has a coating that insulates said wire coil from an external medium over at least a portion of said wire coil.

13. The method according to claim 11, further comprising removing said catheter after step (d).
